(19) United States
(12) Patent Application Publication
(10) Pub. No.: US 2009/0250058 A1
(43) Pub. Date: Oct. 8, 2009

(54) Inhalation System and Delivery Device for the Administration of a Drug in the Form of Dry Powder

(75) Inventors: Orest Lastow, Lund (SE); Lars Arvidsson, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/373,720

(22) PCT Filed: Jul. 12, 2007
(86) PCT No.: PCT/SE07/00682

§ 371 Date: Jan. 13, 2009

Related U.S. Application Data
(60) Provisional application No. 60/831,145, filed on Jul. 14, 2006.

(51) Int. Cl. A61M 15/00

(52) U.S. Cl. 128/203.15

(57) ABSTRACT

The administration of at least one drug in the form of dry powder by inhalation through an air flow path may be accomplished by an inhalation system including a delivery device having a mouthpiece through which the powder is inhaled. The inhalation system includes a dose cassette defining at least one drug cavity containing dry powder of a dose to be delivered and being sealed by a lid. Further, the inhalation system includes a resilient member configured to be introduced into the air flow path to guide/direct the air flow into the drug cavity upon removal of the lid.

### TECHNICAL FIELD

The present invention generally relates to an inhalation system for the administration of a drug/medicament in the form of dry powder by inhalation through an air flow path. The system includes, for example, a delivery device having a mouthpiece through which the powder is inhaled, a dose cassette being sealed by at least one lid and including at least one drug cavity for each dose to be delivered.

### BACKGROUND

Inhalation systems and single dose disposable dry powder inhalation devices may be used for early clinical studies, for production in large scale for regular use of a patient suffering from a respiratory disorder, and/or, if desired, treating a systematic disease through administration of a drug via inhalation.

Available devices with separate doses are capsule based devices, e.g. Spinhaler®, Rotahaler®, FlowCaps® and Cyclohaler®. A capsule is fragile and moisture sensitive and must be stored in individual blister packs. When opening the capsule, the walls are ruptured and fragments of the capsule might be inhaled.

In WO98/34661, a single dose disposable inhaler for administering powder by inhalation is described. The known inhaler comprises a channel through which a stream of air may be drawn by inhalation of a user; and a powder dispenser for providing said powder in said stream of air for inhalation by the user. The channel includes at least one deagglomeration section with a section inlet, a section outlet downstream of said section inlet and a divider between said section inlet and said section outlet for dividing said stream of air either side of said divider. The divider has a surface opposite said section inlet and said surface is oriented at an angle substantially perpendicular to the flow of said stream of air passing through said section inlet.

### SUMMARY

The system disclosed herein may provide higher patient compliance and lower cost per dose. Due to the use of a drug cassette, the system is both durable and robust. In addition, the cassette is suitable for labeling. Fine particle fraction and low retention may be achieved with this system.

In one aspect, the administration of at least one drug in the form of dry powder by inhalation through an air flow path may be accomplished by an inhalation system including a delivery device, a dose cassette, and a resilient member. The delivery device has a mouthpiece through which the powder is inhaled. The dose cassette defines a drug cavity containing dry powder of a dose to be delivered. The drug cavity is sealed by a lid. The resilient member is configured to be introduced into the air flow path to guide/direct the air flow into the drug cavity upon removal of the lid.

Implementations of described features may include one or more of the following features. One or more drug cavities may be surrounded by moisture absorbent material, at least in part. The resilient member may protrude into the drug cavity and, in some implementations, may be adapted to create a turbulent air flow in the drug cavity to remove the powder from the cavity. The delivery device may be a single dose disposable delivery device.

In some implementations, the resilient member may have a triangular end. The resilient member may be formed integrally with the delivery device.

In some implementations, the cassette defines multiple drug cavities containing dry powder to be inhaled simultaneously as a single dose. The drug cavities may be surrounded by moisture absorbent material, at least in part. The drug cavities may contain a single type of drug or different types of drug. In some implementations, the different types of drugs may be incompatible with each other. The drug cavities may have the same size or different size. The two or more drug cavities may be covered by a common lid or, alternatively, each cavity may be covered with its respective lid.

In some implementations, the resilient member is a first resilient member that is introduced into the air flow path to guide/direct air flow into the first drug cavity, and the delivery device includes a second resilient member that is introduced into the air flow path to guide/direct the air flow into the second drug cavity upon removal of a lid sealing the second cavity. In some implementations, the first and second resilient members are introduced substantially simultaneously into the air flow path, whereby the drug in the first drug cavity and in the second drug cavity are inhalable substantially simultaneously.

In another aspect, a single dose disposable delivery device includes a mouthpiece through which at least one drug is inhaled along an air flow path. The deliver device also includes a housing configured to receive a single dose cassette defining a drug cavity containing powder of a dose of medicament to be inhaled and sealed by a lid. The delivery device also includes a resilient member configured to be introduced into the air flow path to guide/direct air flow into the drug cavity upon removal of the lid.

Implementations may include one or more of the following features. The delivery device may include a key adapted to fit a specific cassette being coupled to the delivery device. The delivery device may include an auxiliary spacer, such as a ventilator or holding chamber, coupled to the mouthpiece. In addition, the delivery device may include an auxiliary bellow coupled to the air flow path and arranged to discharge drug aerosol into the spacer.

In some implementations, the resilient member is a first resilient member which is introduced into the air flow path to guide/direct the air flow into a first drug cavity of the cassette, and the delivery device may include a second resilient member which is introduced into the air flow path to guide/direct air flow into a second drug cavity of the cassette upon removal of a lid sealing the second drug cavity. The first and second resilient members may be introduced substantially simultaneously into the air flow path, whereby the drug in the first drug cavity and in the second drug cavity are inhalable substantially simultaneously.

In another aspect, administration of at least one drug in the form of dry powder by inhalation through an air flow path is accomplished by an inhalation system including a delivery device, a dose cassette, and at least one directing member positioned in the air flow path to guide/direct the air flow into the drug cavities upon removal of the at least one lid. The delivery device has a mouthpiece through which the powder is inhaled, and the dose cassette includes for each dose multiple drug cavities containing drug to be inhaled simultaneously as a dose of medicament, the drug cavities being sealed by at least one lid. The drug cavities may be covered by a common lid or, alternatively, each cavity may be covered with its respective lid.

In another aspect, a single dose disposable delivery device includes a mouthpiece through which at least one drug is inhaled along an air flow path from a single dose cassette. The single dose cassette includes at least a first and a second drug cavity comprising the dose to be inhaled and sealed by at least one lid. The delivery device includes a directing member, wherein the directing member is introduced into the air flow path to guide/direct the air flow into the drug cavities upon removal of the at least one lid.

The system described herein is suitable for low volume (<1 k units) manual filling and assembly. The same system is also suitable for production in high volumes (>1 M units) using a fully automated production line.

Furthermore, even though the directing member may optionally be a resilient member as has been described above, the directing member may, as an alternative, be non-resilient.

### DESCRIPTION OF DRAWINGS

FIG. 1 is a schematic overview illustrating a single dose disposable delivery device, here in the form of an inhalation unit.

FIGS. 2a and 2b illustrate details of the inhalation unit.

FIGS. 3a-3d illustrate various cassette configurations.

FIG. 4 illustrates the cassettes placed in a row.

FIGS. 5a-5c show a sequence in which the lidding material is removed from a cassette that has been torn off from the row of cassettes illustrated in FIG. 4.

FIGS. 6a and 6b schematically illustrate details of at least one embodiment of an inhalation unit as an alternative to the one illustrated in FIGS. 2a and 2b.

FIG. 7 schematically illustrates details of at least another embodiment of an inhalation unit.

### DETAILED DESCRIPTION

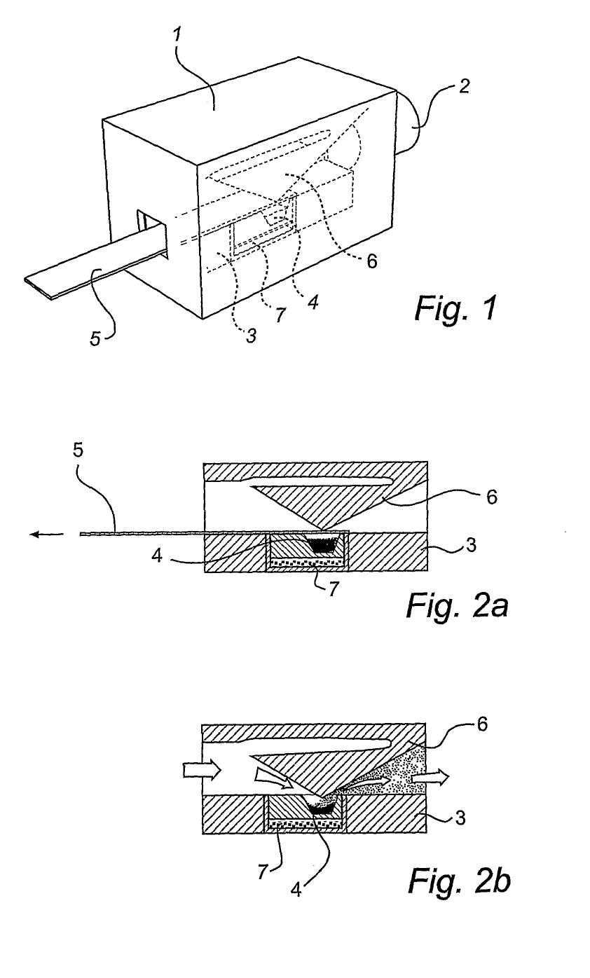

FIG. 1 is a schematic overview illustrating a single dose disposable delivery device 1, here in the form of an inhalation unit, according to at least one example of the present invention. The device includes a mouthpiece 2 through which the user inhales. The inhalation channel is configured to give good performance in terms of fine particle fraction (FPF) around 30% and low retention. The delivery device 1 is a single injection moulded component. A cassette 3 with prefilled drug cavities 4 is loaded into the device. The cassette is injection moulded and includes one or more drug cavities 4 holding the formulation, and a lidding material 5 e.g. Al foil. If needed, inhalation units can be coded to only work with a certain cassette.

FIGS. 2a and 2b illustrate details of the inhalation unit, with a resilient member 6 located above a loaded cassette 3 with a drug cavity 4. In FIG. 2a an arrow indicates that the lidding material 5 covering the drug cavity 4 is to be peeled off. Thereafter, a user may inhale the drug contained in the drug cavity 4. Before the lidding material 5 is peeled off, a portion of the resilient member 6, here illustrated as a tip of the resilient member 6, rests or is biased against the lidding material 5. As shown in FIG. 2b the resilient member 6 is allowed to protrude into the drug cavity 4 after the lidding has been peeled off. The airflow created when the user inhales is thus led into the open drug cavity, enabling the cavity to be emptied of powder, as indicated by the arrows in FIG. 2b. The resilient member 6 is preferably designed to create a turbulent airflow when air enters the drug cavity 4. This is to achieve an efficient emptying of the drug cavity 4. The inhalation unit can accommodate any suitable cassette filled with any suitable formulation, drug, dose size etc. If needed the inhalation units can be coded to only work with a certain cassette, e.g. the inhalation unit may include a key which is adapted to fit a specific cassette.

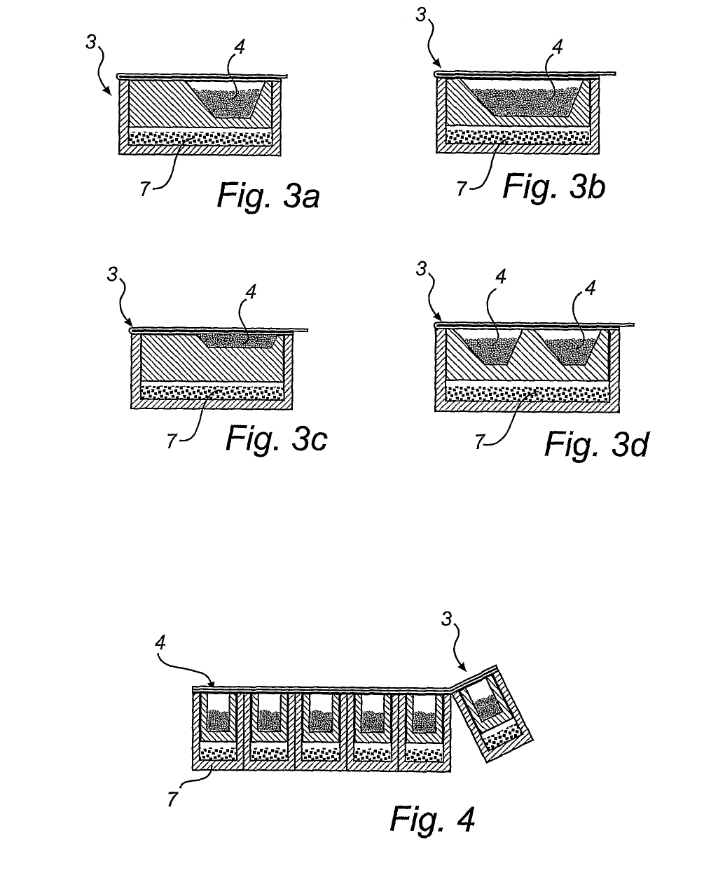

FIGS. 3a-3d illustrate details of various cassette configurations, with different shape and size of drug cavities 4. The cassettes 3 are injection moulded and include one or more drug cavities holding the drug to be inhaled, and a lidding material, e.g. Al foil, which is folded in two layers. When the cassette has more than one drug cavity, several chemically incompatible drugs can be inhaled simultaneously to provide a combinatory effect. The cassettes 3 can be filled either manually or by using commercial dosating fillers. A simple bench top filling equipment can be used for small series down to about 1 g of formulation. The preferred formulation is a carrier-based formulation but also a pure micronised powder can be used. After the cassettes are filled with the drug, the cassettes are sealed using conventional heat sealing. The cassettes can now be distributed and stored separate from the inhalation unit. The cassette 3 can have dual walls with a desiccant 7 in between. For extra high moisture protection, cassettes with moisture protection of the type described in WO2006/00758 can be used. The drug cavities 4 in the cassette can be shaped for different filling volumes or types of formulation. The filling weigh can be from 500 ug to 30 mg, preferably from 1 mg to 20 mg, and most preferably from 1 mg to 15 mg. The cassette 3 can have more than one drug cavity to accommodate several chemically incompatible drugs (see e.g. FIG. 3d) and the different formulations will be inhaled simultaneously to give a combinatory effect.

Each cassette can either be provided as a separate unit or be provided as a set of cassettes, the latter being illustrated in FIG. 4.

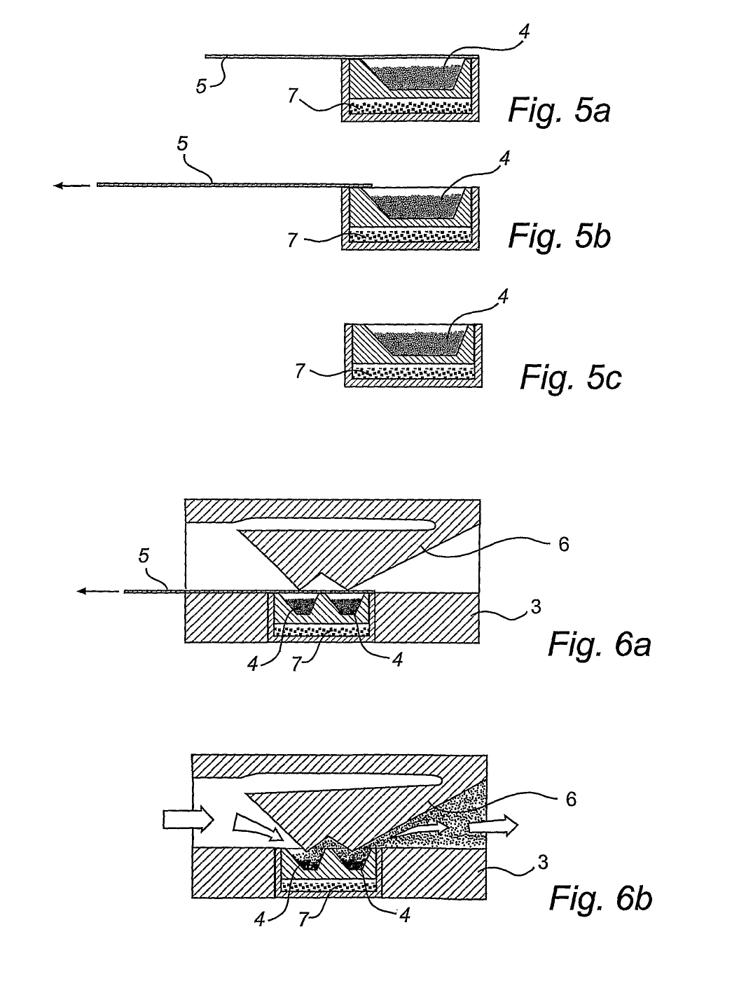

FIG. 4 illustrates the cassettes 3 placed in a row. The lidding material is folded in two layers (as illustrated in FIGS. 3a-3d). A cassette 3 is torn off, suitably along a perforated line, from the row of cassettes. Next, as illustrated in FIG. 5a, the lidding material 5 in the shape of a strap on the cassette is folded back before the cassette is inserted into a delivery device which is then closed. The delivery device is then locked and cannot be opened. The strap extends out of the delivery device and can easily be pulled off. By pulling of the strap (FIG. 5b), the formulation is exposed and the device is ready for inhalation (FIG. 5c). During inhalation the air is forced through the drug cavity by a resilient member that bends down into the drug cavity after removing the foil. After inhaling, the complete system is disposed. All retained drug, if any, is contained inside the device and cannot be accessed by the user. Further, by disposing the system after use, the problem with repeated retention disturbing the system to give a correct dose is avoided. The system can be fitted with an auxiliary bellow to actively discharge the aerosol into a ventilator or spacer.

The use of two drug cavities illustrated in FIG. 3d is further illustrated in FIGS. 6a and 6b, which show details of at least one embodiment of an inhalation unit as an alternative to the one illustrated in FIGS. 2a and 2b. As can be seen in FIGS. 6a and 6b, when the lidding material 5 is torn off, two resilient members 6 (here shown as formed in one piece) are enabled to protrude into their respective associated drug cavity 4, whereby both drugs may be inhaled simultaneously.

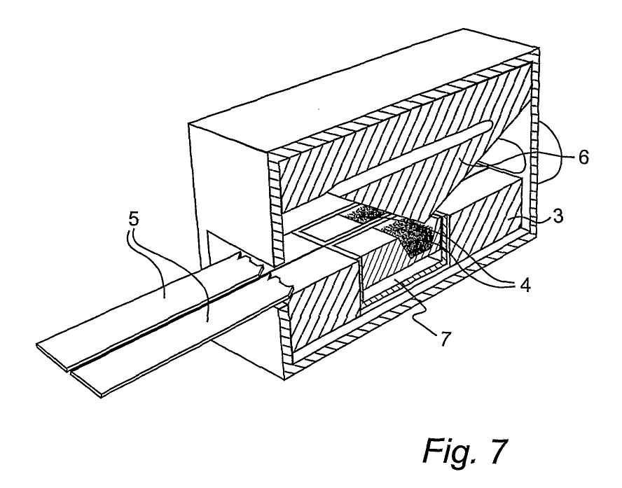

FIG. 7 schematically illustrates details of at least another embodiment of an inhalation unit. For clarity purposes, the inhalation unit is shown in a perspective view and partially in cross-section. While FIGS. 6a and 6b illustrate the two cavities being serially arranged, FIG. 7 illustrates two cavities being arranged in parallel. A lidding material 5 in the form of a single strap may cover both cavities, or as illustrated in FIG. 7, two straps 5 may cover a respective cavity, thereby allowing the user to uncover one or both cavities before inhalation. By uncovering one or both cavities, the user is thereby allowed to select how large dose to inhale (if the same drug is present in both cavities), or which drug or drugs to inhale (if different drugs are present in the two cavities). It should be noted that the schematic illustrations in FIGS. 2a and 2b may also represent a cross-sectional view of an inhalation unit according to FIG. 7.

The delivery device 1 may be used with any suitable form of powder, including powders introduced into the air stream in the raw state or as agglomerate, micronised or carrier based formulation. Furthermore, the active ingredient or ingredients of the powder may be diluted with one or more substances such as lactose and may include substances for the treatment of various conditions, not necessarily respiratory conditions. Indeed, the powder can include genetic material and need not be restricted to human use only.

Drugs suitable for administration by the powder delivery device 1 are any which may be delivered by inhalation and include for example β2-adrenoreceptor agonists, for example, salbutamol, terbutaline, rimiterol, fenoterol, reproterol, adrenaline, pirbuterol, isoprenaline, orciprenaline, bitolterol, salmeterol, formoterol, clenbuterol, procaterol, broxaterol, picumeterol, TA-2005, mabuterol and the like, and their pharmacologically acceptable esters and salts; anticholinergic bronchodilators, for example, ipratropium bromide and the like; glucocorticosteroids, for example, beclomethasone, fluticasone, budesonide, tipredane, dexamethasone, betamethasone, fluocinolone, triamcinolone acetonide, mometasone and the like, and their pharmacologically acceptable esters and salts; antiallergic drugs, for example, sodium cromoglycate and nedocromil sodium; expectorants; mucolytics; antihistamines; cyclooxygenase inhibitors; leukotriene synthesis inhibitors; leukotriene antagonists; phospholipase-A2 (PLA2) inhibitors; platelet aggregating factor (PAF) antagonists and prophylactics of asthma; antiarrhythmic drugs; tranquilisers; cardiac glycosides; hormones; antihypertensive drugs; antidiabetic drugs; antiparasitic drugs; anticancer drugs; sedatives; analgesic drugs; antibiotics; antirheumatic drugs; immunotherapies; antifungal drugs; antihypotension drugs; vaccines; antiviral drugs; proteins; polypeptides and peptides, for example, peptide hormones and growth factors; polypeptide vaccines; enzymes; endorphines; lipoproteins and polypeptides involved in the blood coagulation cascade; vitamins; and others, for example, cell surface receptor blockers, antioxidants, free radical scavengers and organic salts of N,N′-diacetylcystine.

Suitable glucocorticosteroids include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters according to WO 2002/12265, WO 2002/12266 and WO 2002/88167 e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3 S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, steroids according to WO 2005/041980, steroids GSK 870086, GSK 685698, GSK 799943 and the like.

Preferably the bronchodilator is a long-acting P₂-agonist. Suitable long-acting β₂-agonists include salmeterol, formoterol, bambuterol, TA 2005 (chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854 (=CHF-4226, carmoterol)), QAB149 (CAS no 312753-06-3; indacaterol), GSK 159797, formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)-benzenesulfonamide as disclosed in WO 2002/88167, aryl aniline receptor agonists as disclosed in WO 2003/042164 and WO 2005/025555, indole derivatives as disclosed in WO 2004/032921 and the like.

Among the anticholinergic compounds may be mentioned ipratropium (e.g. as bromide), tiotropium (e.g. as bromide), oxitropium (e.g. as bromide), tolterodine, AD-237 (Arakis), quinuclidine derivatives as disclosed in US 2003/0055080 and the like. Several of these compounds could be administered in the form of pharmacologically acceptable esters, salts, solvates, such as hydrates, or solvates of such esters or salts, if any. Both racemic mixtures as well as one or more optical isomers of the above compounds may be used with the present invention.

The invention is not limited only to the embodiments described above and shown in the drawings. Thus, the system as well as the delivery device may be modified in all kinds of ways within the scope of the appended claims.

1. An inhalation system for the administration of at least one drug in the form of dry powder by inhalation through an air flow path comprising:
a delivery device having a mouthpiece through which the powder is inhaled;
a dose cassette defining a drug cavity containing dry powder of a dose to be delivered and being sealed by a lid; and
a resilient member configured to be introduced into the air flow path to direct air flow into the drug cavity upon removal of the lid.

2. The inhalation system of claim 1 wherein said at the drug cavity is at least partly surrounded by moisture absorbent material.

3. The inhalation system of claim 1 wherein the resilient member protrudes into the drug cavity.

4. The inhalation system of claim 3 wherein the resilient member is adapted to create a turbulent air flow in the drug cavity to remove the powder therefrom.

5. The inhalation system of claim 1 wherein the delivery device is a single dose disposable delivery device.

6. The inhalation system of claim 1 wherein the resilient member has a triangular end.

7. The inhalation system of claim 1 wherein the resilient member is formed integrally with the delivery device.

8. The inhalation system of claim 1 wherein the cassette defines multiple drug cavities containing dry powder to be inhaled simultaneously as a single dose.

9. The inhalation system of claim 8 wherein the drug cavities are at least partly surrounded by moisture absorbent material.

10. The inhalation system of claim 8 wherein the drug cavities contain a single type of drug.

11. The inhalation system of claim 8 wherein the drug cavities contain different types of drug.

12. The inhalation system of claim 11 wherein the different types of drugs are compatible with each other.

13. The inhalation system of claim 8 wherein the cavities are of the same size.

14. The inhalation system of claim 8 wherein the drug cavities are of different size.

15. The inhalation system of claim 8 wherein the multiple drug cavities include a first and second drug cavity and wherein the resilient member is a first resilient member that is introduced into the air flow path to direct air flow into the first drug cavity, the delivery device further comprising a second resilient member that is introduced into the air flow path to direct air flow into the second drug cavity upon removal of a lid sealing the second drug cavity.

16. The inhalation system of claim 15 wherein the first and second resilient members are introduced substantially simultaneously into the air flow path, whereby the drug in the first drug cavity and in the second drug cavity are inhalable substantially simultaneously.

17. A single dose disposable delivery device comprising a mouthpiece through which at least one drug is inhaled along an air flow path and further comprising:
a housing configured to receive a single dose cassette defining a drug cavity containing powder of a dose of medicament to be inhaled and sealed by a lid; and
a resilient member configured to be introduced into the air flow path to direct air flow into the drug cavity upon removal of the lid.

18. The delivery device of claim 17 further comprising a key adapted to fit a specific cassette being coupled to a the delivery device.

19. The delivery device of claim 17 further comprising an auxiliary spacer coupled to the mouthpiece.

20. The delivery device of claim 19 wherein the spacer is a ventilator.

21. The delivery device of claim 19 wherein said spacer is a holding chamber.

22. The delivery device of claim 19 further comprising an auxiliary bellow arranged to discharge drug aerosol into the spacer.

23. The delivery device of claim 17 wherein the resilient member is a first resilient member which is introduced into the air flow path to direct air flow into a first drug cavity of the cassette, the delivery device further comprising a second resilient member which is introduced into the air flow path to direct air flow into a second drug cavity of the cassette upon removal of a lid sealing the second drug cavity.

24. The delivery device of claim 23 wherein the first and second resilient members are introduced substantially simultaneously into the air flow path, whereby the drug in the first drug cavity and in the second drug cavity are inhalable substantially simultaneously.

25. An inhalation system for the administration of at least one drug in the form of dry powder by inhalation through an air flow path, the system comprising:
a delivery device, having a mouthpiece through which the powder is inhaled,
a dose cassette comprising for each dose multiple drug cavities containing drug to be inhaled simultaneously as a dose of medicament, the drug cavities being sealed by at least one lid; and
at least one directing member positioned
in the air flow path to direct air flow into the drug cavities upon removal of the at least one lid.

26. A single dose disposable delivery device, comprising a mouthpiece through which at least one drug is inhaled along an air flow path from a single dose cassette comprising at least a first and a second drug cavity comprising the dose to be inhaled and sealed by at least one lid,
the device further comprising a directing member wherein the directing member is introduced into the air flow path to direct air flow into the drug cavities upon removal of the at least one lid.
